SEQUENCE LISTING (69 sequences)

SEQ ID NO: 1 - RNA, 22 nt, artificial
  oligonucleotide or target sequence
ugugacugguugaccagagggg

SEQ ID NO: 2 - RNA, 19 nt, artificial
  oligonucleotide or target sequence
gugacugguugaccagagg

SEQ ID NO: 3 - RNA, 18 nt, artificial
  oligonucleotide or target sequence
gugacugguugaccagag

SEQ ID NO: 4 - DNA, 18 bp, artificial
  oligonucleotide or target sequence
ctctggtcaaccagtcac

SEQ ID NO: 5 - DNA, 18 bp, artificial
  oligonucleotide or target sequence
ctctggtcaaccagtcac

SEQ ID NO: 6 - DNA, 18 bp, artificial
  oligonucleotide or target sequence
ctctggtcaaccagtcac

SEQ ID NO: 7 - DNA, 18 bp, artificial
  oligonucleotide or target sequence
ctctggtcaaccagtcac

SEQ ID NO: 8 - DNA, 18 bp, artificial
  oligonucleotide or target sequence
ctctggtcaaccagtcac

SEQ ID NO: 9 - DNA, 18 bp, artificial
  oligonucleotide or target sequence
ctctggtcaaccagtcac

SEQ ID NO: 10 - DNA, 18 bp, artificial
  oligonucleotide or target sequence
ctctggtcaaccagtcac

SEQ ID NO: 11 - DNA, 18 bp, artificial
  oligonucleotide or target sequence
ctctggtcaaccagtcac

SEQ ID NO: 12 - DNA, 18 bp, artificial
  oligonucleotide or target sequence
ctctggtcaaccagtcac

SEQ ID NO: 13 - DNA, 18 bp, artificial
  oligonucleotide or target sequence
ctctggtcaaccagtcac

SEQ ID NO: 14 - DNA, 18 bp, artificial
  oligonucleotide or target sequence
ctctggtcaaccagtcac

SEQ ID NO: 15 - DNA, 18 bp, artificial
  oligonucleotide or target sequence
ctctggtcaaccagtcac

SEQ ID NO: 16 - DNA, 18 bp, artificial
  oligonucleotide or target sequence
ctctggtcaaccagtcac

SEQ ID NO: 17 - DNA, 19 bp, artificial
  oligonucleotide or target sequence
cctctggtcaaccagtcac

SEQ ID NO: 18 - DNA, 19 bp, artificial
  oligonucleotide or target sequence
cctctggtcaaccagtcac

SEQ ID NO: 19 - DNA, 19 bp, artificial
  oligonucleotide or target sequence
cctctggtcaaccagtcac

SEQ ID NO: 20 - DNA, 18 bp, artificial
  oligonucleotide or target sequence
ctctggtcaaccagtcac

SEQ ID NO: 21 - DNA, 19 bp, artificial
  oligonucleotide or target sequence
cctctggtcaaccagtcac

SEQ ID NO: 22 - DNA, 15 bp, artificial
  oligonucleotide or target sequence
tggtcaaccagtcac

SEQ ID NO: 23 - DNA, 18 bp, artificial
  oligonucleotide or target sequence
tctggtcaaccagtcaca

SEQ ID NO: 24 - DNA, 18 bp, artificial
  oligonucleotide or target sequence
tctggtcaaccagtcaca

SEQ ID NO: 25 - DNA, 18 bp, artificial
  oligonucleotide or target sequence
tctggtcaaccagtcaca

SEQ ID NO: 26 - DNA, 18 bp, artificial
  oligonucleotide or target sequence
tctggtcaaccagtcaca

SEQ ID NO: 27 - DNA, 19 bp, artificial
  oligonucleotide or target sequence
ctctggtcaaccagtcaca

SEQ ID NO: 28 - DNA, 19 bp, artificial
  oligonucleotide or target sequence
ctctggtcaaccagtcaca

SEQ ID NO: 29 - DNA, 19 bp, artificial
  oligonucleotide or target sequence
ctctggtcaaccagtcaca

SEQ ID NO: 30 - DNA, 19 bp, artificial
  oligonucleotide or target sequence
ctctggtcaaccagtcaca

SEQ ID NO: 31 - DNA, 17 bp, artificial
  oligonucleotide or target sequence
tctggtcaaccagtcac

SEQ ID NO: 32 - DNA, 17 bp, artificial
  oligonucleotide or target sequence
tctggtcaaccagtcac

SEQ ID NO: 33 - DNA, 17 bp, artificial
  oligonucleotide or target sequence
tctggtcaaccagtcac

SEQ ID NO: 34 - DNA, 17 bp, artificial
  oligonucleotide or target sequence
tctggtcaaccagtcac

SEQ ID NO: 35 - DNA, 19 bp, artificial
  oligonucleotide or target sequence
cctctggtcaaccagtcac

SEQ ID NO: 36 - DNA, 19 bp, artificial
  oligonucleotide or target sequence
cctctggtcaaccagtcac

SEQ ID NO: 37 - DNA, 19 bp, artificial
  oligonucleotide or target sequence
cctctggtcaaccagtcac

SEQ ID NO: 38 - DNA, 17 bp, artificial
  oligonucleotide or target sequence
ctctggtcaaccagtca

SEQ ID NO: 39 - DNA, 17 bp, artificial
  oligonucleotide or target sequence
ctctggtcaaccagtca

SEQ ID NO: 40 - DNA, 17 bp, artificial
  oligonucleotide or target sequence
ctctggtcaaccagtca

SEQ ID NO: 41 - DNA, 17 bp, artificial
  oligonucleotide or target sequence
ctctggtcaaccagtca

SEQ ID NO: 42 - DNA, 17 bp, artificial
  oligonucleotide or target sequence
ctctggtcaaccagtca

SEQ ID NO: 43 - DNA, 17 bp, artificial
  oligonucleotide or target sequence
ctctggtcaaccagtca

SEQ ID NO: 44 - DNA, 17 bp, artificial
  oligonucleotide or target sequence
ctctggtcaaccagtca

SEQ ID NO: 45 - DNA, 18 bp, artificial
  oligonucleotide or target sequence
cctctggtcaaccagtca

SEQ ID NO: 46 - DNA, 18 bp, artificial
  oligonucleotide or target sequence
cctctggtcaaccagtca

SEQ ID NO: 47 - DNA, 18 bp, artificial
  oligonucleotide or target sequence
cctctggtcaaccagtca

SEQ ID NO: 48 - DNA, 18 bp, artificial
  oligonucleotide or target sequence
cctctggtcaaccagtca

SEQ ID NO: 49 - DNA, 18 bp, artificial
  oligonucleotide or target sequence
cctctggtcaaccagtca

SEQ ID NO: 50 - DNA, 18 bp, artificial
  oligonucleotide or target sequence
cctctggtcaaccagtca

SEQ ID NO: 51 - DNA, 18 bp, artificial
  oligonucleotide or target sequence
cctctggtcaaccagtca

SEQ ID NO: 52 - DNA, 19 bp, artificial
  oligonucleotide or target sequence
cctctggtcaaccagtcac

SEQ ID NO: 53 - DNA, 19 bp, artificial
  oligonucleotide or target sequence
cctctggtcaaccagtcac

SEQ ID NO: 54 - DNA, 18 bp, artificial
  oligonucleotide or target sequence
ctctggtcaaccagtcac

SEQ ID NO: 55 - DNA, 18 bp, artificial
  oligonucleotide or target sequence
ctctggtcaaccagtcac

SEQ ID NO: 56 - DNA, 18 bp, artificial
  oligonucleotide or target sequence
ctctggtcaaccagtcac

SEQ ID NO: 57 - DNA, 18 bp, artificial
  oligonucleotide or target sequence
ctctggtcaaccagtcac

SEQ ID NO: 58 - DNA, 18 bp, artificial
  oligonucleotide or target sequence
ctctggtcaaccagtcac

SEQ ID NO: 59 - DNA, 18 bp, artificial
  oligonucleotide or target sequence
ctctggtcaaccagtcac

SEQ ID NO: 60 - DNA, 19 bp, artificial
  oligonucleotide or target sequence
cctctggtcaaccagtcac

SEQ ID NO: 61 - DNA, 22 bp, Artificial Sequence
  Limk1 forward primer
ggtagcatcaggtattgacagg

SEQ ID NO: 62 - DNA, 20 bp, Artificial Sequence
  Reverse primer Limk1
agatggacacgaagcaagag

SEQ ID NO: 63 - DNA, 22 bp, Artificial Sequence
  Forward primer Serpine1
cctcttgtatcgtcctccattg

SEQ ID NO: 64 - DNA, 21 bp, Artificial Sequence
  Reverse primer Serpine1
gatgggcaaggtgttcatttc

SEQ ID NO: 65 - DNA, 23 bp, Artificial Sequence
  Forward primer Hprt1
ggagaacaattctgggtttgatc

SEQ ID NO: 66 - DNA, 20 bp, Artificial Sequence
  Reverse primer Hprt1
tgtgaagttccccataaggc

SEQ ID NO: 67 - DNA, 25 bp, Artificial Sequence
  Limk1 probe labelled with 56-FAM, ZEN and 3IABkFQ in accordance with Table 2.
tcccaaagcagagagagagagaggt

SEQ ID NO: 68 - DNA, 24 bp, Artificial Sequence
  Serpine1 probe labelled with 56-FAM, ZEN and 3IABkFQ in accordance with Table 2.
ttcttcctttggcctgggtcatcc

SEQ ID NO: 69 - DNA, 23 bp, Artificial Sequence
  Hprt probe labelled with 56-FAM, ZEN and 3IABkFQ in accordance with Table 2.
tgttgacccaccagcagttcagt

(19) United States
(12) Patent Application Publication
(10) Pub. No.: US 2024/0301414 A1
(43) Pub. Date: Sep. 12, 2024

(54) MICRORNA-134 INHIBITORS

(71) Applicant: NEUmiRNA Therapeutics Aps, København SV (DK)

(72) Inventors: Markus Sakari Kauppinen, Holte (DK); Stine Normann Hansen, København S (DK); Henrik Valdemar Klitgaard, Hellerup (DK)

(21) Appl. No.: 18/552,335

(22) PCT Filed: Mar. 28, 2022
(86) PCT No.: PCT/EP2022/058140

§ 371 Date: Sep. 25, 2023

(30) Foreign Application Priority Data
Mar. 26, 2021 (DK) PA 2021 70145

(51) Int. Cl. C12N 15/113

(52) U.S. Cl. CPC C12N 15/113; C12N 2310/113; C12N 2310/315; C12N 2310/3231; C12N 2310/3341

(57) ABSTRACT

The present invention provides novel highly potent antisense oligonucleotides. Such compounds are useful for making pharmaceutical compositions for treatment of a range of diseases where modulation of miR-134 activity is beneficial, including neurological diseases such as epilepsy.

### FIELD OF THE INVENTION

The present invention relates to new compounds and compositions capable of inhibiting the activity of microRNA-134 (miR-134). In particular, the invention provides antisense oligonucleotide compounds capable of modulating the activity of miR-134 in a human in vivo, useful for treating CNS disorders, including epilepsy.

### BACKGROUND

Epilepsy is a serious, chronic neurological disorder characterised by recurrent spontaneous seizures affecting about 50 million people worldwide.

Present anti-epileptic drugs that are available, typically control seizures in two-thirds of patients, but probably have no effect on the underlying pathophysiology. The remaining one-third of patients with epilepsy are either drug resistant or suffer from serious side effects from the presently available drugs.

An alternative to avoiding seizures in patients without the option of getting drug treatment is ketogenic diet, brain surgery, vagus nerve and intra cranial stimulation.

The development of symptomatic (acquired) epilepsy is thought to involve altered expression of ion channels and neurotransmitter receptors, synaptic remodelling, inflammation, gliosis and neuronal death, among others. However, our understanding of the cell and molecular mechanisms remains incomplete. There are currently no prophylactic treatments (“anti-epileptogenic”) following a brain injury likely to precipitate epilepsy. Similarly, there is no specific neuroprotective treatment for status epilepticus (SE), or treating acute neurologic injuries likely to cause brain damage or epilepsy, for example, stroke, or trauma.

Recent data suggest that microRNAs (miRNAs) are critical to the pathogenesis of several neurologic disorders, including epilepsy. MiRNAs comprise a class of short (˜22 nt) endogenous non-coding RNAs that mediate post-transcriptional regulation of gene expression (Ambros, 2004 Nature, (2004) Sep. 16; 431(7006):350-5; Bartel, 2009 Cell, (2009) Jan. 23; 136(2): 215-33). Mature miRNAs serve as guide molecules for the miRISC complex by directing it to partially complementary target sites located predominantly in the 3′ untranslated regions (UTRs) of target mRNAs, resulting in translational repression and/or mRNA degradation of the targets (van Rooij & Kauppinen EMBO Mol Med (2014), July; 6(7): 851-64). An important determinant guiding miRNA target recognition is the base pairing of the miRNA seed region (nucleotides 2-7 in the mature miRNA) with a perfectly complementary seed match site in the target mRNA 3′ UTR (Bartel, 2009 Cell, (2009) Jan. 23; 136(2): 215-33). MicroRNA-134 (miR-134) is a brain-specific, activity-regulated miRNA implicated in the control of neuronal microstructure. Pyramidal cells are the most common neurons in the neocortex and hippocampal formation. They are the major source of intrinsic excitatory cortical synapses, and their dendritic spines are the main postsynaptic target of excitatory synapses, with spine size an index of synaptic strength. In the adult brain, spines are quite stable, but remodelling occurs during learning and memory formation, as well in the setting of neuropsychiatric disorders and pathological brain activity. Spine collapse is mediated in part by N-methyl-D-aspartate (NMDA) receptor/calcium-dependent de-polymerisation of actin by cofilin. LIM kinase-1 (Limk1) phosphorylates and inactivates cofilin and loss of Limk1 results in abnormal spine morphology. In hippocampal neurons, miR-134 targets Limk1 mRNA, thereby preventing Limk1 protein translation. Over-expression of miR-134 in vitro has been reported to reduce spine volume, whereas over-expression of miR-134 in vivo using viral vectors reduces total dendritic length and abrogates long-term potentiation (LTP). Mice lacking the miRNA biogenesis component Dgrc8 fail to produce several mature miRNAs, including miR-134, and display reduced hippocampal spine density. Spine loss may have divergent functional consequences according to context, promoting excitability or uncoupling NMDA receptor-driven currents in neurons and preventing excitotoxicity. Silencing of miR-134 expression in vivo using antimiRs reduced hippocampal CA3 pyramidal neuron dendrite spine density by 21% and rendered mice refractory to seizures and hippocampal injury caused by status epilepticus. Depletion of miR-134 after status epilepticus in mice reduced the later occurrence of spontaneous seizures by over 90% and mitigated the attendant pathological features of temporal lobe epilepsy. Thus, inhibition of miR-134 activity leads to prolonged suppression of seizures and increased neuroprotection.

In summary, there is still a significant unmet need for new efficacious and safe therapies useful as treatment or a preventative measure that specifically targets the process by which epilepsy and other neurological injuries likely to cause brain damage develop and that overcome some of the above-mentioned problems.

### SUMMARY OF THE INVENTION

The present invention provides novel compounds that are potent inhibitors of microRNA 134 (miR-134). Such compounds are useful in compositions for medical use, such as for treatment, alleviation, amelioration, pre-emptive treatment or prophylaxis of diseases where modulation of miR-134 is beneficial. Such diseases comprise neurological diseases, including epilepsy and memory disorders.

The compounds of the invention are antisense oligonucleotides complementary to miR-134 (SEQ ID NO 1) comprising a sequence of 18-19 nucleotides in length, wherein the antisense oligonucleotides are LNA/DNA mixmers and do not contain a stretch of more than three contiguous DNA nucleotides, and wherein said antisense oligonucleotides harbor between one and 18 phosphorothioate internucleoside linkages. The antisense oligonucleotides of the invention are complementary to the miR-134 sequence 5′ UGUGACUGGUUGACCAGAGGGG 3′ (SEQ ID NO: 1).

In some embodiments, the invention provides antisense oligonucleotides designed to target part of or the whole of 5′ GUGACUGGUUGACCAGAGG 3′ (SEQ ID NO: 2).

In some embodiments, the antisense oligonucleotides of the invention are designed to target at least 5′ GUGACUGGUUGACCAGAG 3′ (SEQ ID NO: 3).

In some embodiments, the antisense oligonucleotides comprise the sequence 5′CTCTGGTCAACCAGTCAC3′ (SEQ ID NO: 4).

In some embodiments, the antisense oligonucleotide is 18 or 19 nucleotides in length, and comprises the sequence 5′CTCTGGTCAACCAGTCAC3′ (SEQ ID NO: 4).

In some embodiments, the antisense oligonucleotide is 18 or 19 nucleotides in length, comprises the sequence 5′CTCTGGTCAACCAGTCAC3′ (SEQ ID NO: 4) and is a mixmer.

In some embodiments, the antisense oligonucleotide is 18 or 19 nucleotides in length, comprises the sequence 5′CTCTGGTCAACCAGTCAC3′ (SEQ ID NO: 4) and is a LNA/DNA mixmer.

In some embodiments, the antisense oligonucleotide is 18 or 19 nucleotides in length, comprises the sequence 5′CTCTGGTCAACCAGTCAC3′ (SEQ ID NO: 4) and is a LNA/DNA mixmer having between 50 and 70% LNA, such as between 55 and 65% LNA, such as between 55 and 61% LNA, such as at least 50% LNA, such as at least 55% LNA.

In some embodiments, the antisense oligonucleotide is 18 or 19 nucleotides in length, comprises the sequence 5′CTCTGGTCAACCAGTCAC3′ (SEQ ID NO: 4) and is a LNA/DNA mixmer having between 50 and 70% LNA, such as between 55 and 65% LNA, such as between 55 and 61% LNA, such as at least 50% LNA, such as at least 55% LNA, and wherein the two nucleotides in each end are LNA.

In some embodiments, the antisense oligonucleotide is anyone of SEQ ID NO's 5-21 or 23-60.

In some embodiments, the antisense oligonucleotides of the invention (such as those in the above embodiments, or such as SEQ ID NO's: 5-21 or 23-60) are for use as a medicament.

In some embodiments, the antisense oligonucleotide according to the invention consist of anyone of SEQ ID NO's 5-21 or 23-60, optionally comprising a delivery vehicle.

In a preferred embodiment, the antisense oligonucleotide according to the invention comprises SEQ ID NO 7.

In another preferred embodiment, the antisense oligonucleotide according to the invention comprises SEQ ID NO 8.

In another preferred embodiment, the antisense oligonucleotide according to the invention comprises SEQ ID NO 10.

In another preferred embodiment, the antisense oligonucleotide according to the invention comprises SEQ ID NO 12.

In another preferred embodiment, the antisense oligonucleotide according to the invention comprises SEQ ID NO 19.

In another preferred embodiment, the antisense oligonucleotide according to the invention comprises SEQ ID NO 24

In another preferred embodiment, the antisense oligonucleotide according to the invention comprises SEQ ID NO 26

In another preferred embodiment, the antisense oligonucleotide according to the invention comprises SEQ ID NO 27

In another preferred embodiment, the antisense oligonucleotide according to the invention comprises SEQ ID NO 28

In another preferred embodiment, the antisense oligonucleotide according to the invention comprises SEQ ID NO 29

In another preferred embodiment, the antisense oligonucleotide according to the invention comprises SEQ ID NO 30

In another preferred embodiment, the antisense oligonucleotide according to the invention comprises SEQ ID NO 31

In another preferred embodiment, the antisense oligonucleotide according to the invention comprises SEQ ID NO 32

In another preferred embodiment, the antisense oligonucleotide according to the invention comprises SEQ ID NO 33

In another preferred embodiment, the antisense oligonucleotide according to the invention comprises SEQ ID NO 34

In another preferred embodiment, the antisense oligonucleotide according to the invention comprises SEQ ID NO 35

In another preferred embodiment, the antisense oligonucleotide according to the invention comprises SEQ ID NO 36

In another preferred embodiment, the antisense oligonucleotide according to the invention comprises SEQ ID NO 37

In another preferred embodiment, the antisense oligonucleotide according to the invention comprises SEQ ID NO 45

In another preferred embodiment, the antisense oligonucleotide according to the invention comprises SEQ ID NO 46

In another preferred embodiment, the antisense oligonucleotide according to the invention comprises SEQ ID NO 47

In another preferred embodiment, the antisense oligonucleotide according to the invention comprises SEQ ID NO 48

In another preferred embodiment, the antisense oligonucleotide according to the invention comprises SEQ ID NO 49

In another preferred embodiment, the antisense oligonucleotide according to the invention comprises SEQ ID NO 50

In another preferred embodiment, the antisense oligonucleotide according to the invention comprises SEQ ID NO 51

In another preferred embodiment, the antisense oligonucleotide according to the invention comprises SEQ ID NO 52

In another preferred embodiment, the antisense oligonucleotide according to the invention comprises SEQ ID NO 53

In another preferred embodiment, the antisense oligonucleotide according to the invention comprises SEQ ID NO 54

In another preferred embodiment, the antisense oligonucleotide according to the invention comprises SEQ ID NO 55

In another preferred embodiment, the antisense oligonucleotide according to the invention comprises SEQ ID NO 56

In another preferred embodiment, the antisense oligonucleotide according to the invention comprises SEQ ID NO 59.

In another preferred embodiment, the antisense oligonucleotide according to the invention comprises SEQ ID NO 60.

In some embodiments, the invention provides pharmaceutical composition comprising an effective dosage of the antisense oligonucleotides of the invention.

In some embodiments, the antisense oligonucleotide of the invention is for delivery to the brain.

In some embodiments, the antisense oligonucleotide of the invention is for treatment of diseases where modulation of miR-134 activity is beneficial.

In some embodiments, the antisense oligonucleotide of the invention is for treatment of a neurological disease where miR-134 modulation is beneficial.

In some embodiments, the antisense oligonucleotide of the invention is for treatment of epilepsy. The present invention provides highly potent antisense oligonucleotides complementary to microRNA-134 (miR-134), methods for using the antisense oligonucleotides and compositions comprising such antisense oligonucleotides for the treatment of diseases where modulation of miR-134 activity is beneficial, including neurological diseases.

The invention provides highly potent antisense oligonucleotide compounds, and compositions comprising such compounds, as well as medical uses of those for treatment of a variety of diseases where modulation of miR-134 is beneficial.

There is a need for the compounds of the invention, as many of the aforementioned diseases cannot be treated in a sufficient manner, and/or where presently available treatments cause serious side effects.

### DETAILED DESCRIPTION OF THE INVENTION

In describing the embodiments of the invention, specific terminology will be resorted to for the sake of clarity. However, the invention is not intended to be limited to the specific terms so selected, and it is understood that each specific term includes all technical equivalents, which operate in a similar manner to accomplish a similar purpose.

The term “therapeutically effective amount”, or “effective amount” or effective dose”, refers to an amount of a therapeutic agent, which confers a desired therapeutic effect on an individual in need of the agent. The effective amount may vary among individuals depending on the health and physical condition of the individual to be treated, the taxonomic group of the individuals to be treated, the formulation of the composition, the method of administration, assessment of the individual's medical condition, and other relevant factors.

The term “treatment” refers to any administration of a therapeutic medicament, herein comprising an antisense oligonucleotide that partially or completely cures or reduces one or more symptoms or features of a given disease.

The term “compound” as used herein, refers to a compound comprising an oligonucleotide according to the invention. In some embodiments, a compound may comprise other elements apart from the oligonucleotide of the invention. Such other elements may in non-limiting example be a delivery vehicle which is conjugated or in other way bound to the oligonucleotide.

“Antisense oligonucleotide” means a single-stranded oligonucleotide having a nucleobase sequence that permits hybridization to a corresponding region or segment of a target nucleic acid. The antisense oligonucleotide of the present invention is preferably a “mixmer”.

The term “antisense oligonucleotides complementary to miR-134” is used interchangeably with the terms “antimiR-134” or “antimiR-134 oligonucleotide” or “antimiR-134 antisense oligonucleotide”.

A “mixmer” is an antisense oligonucleotide, comprising a mix of nucleoside analogues such as LNA and DNA nucleosides, and wherein the antisense oligonucleotide does not comprise an internal region having a plurality of nucleosides (such as a region of at least 6 or 7 DNA nucleotides), capable of recruiting an RNAse, such as RNAseH, wherein the nucleosides comprising the internal region are chemically distinct from the nucleoside or nucleosides comprising the external wings.

“Nucleoside analogues” are described by e.g. Freier & Altmann; Nucl. Acid. Res., 1997, 25, 4429-4443 and Uhlmann; Curr. Opinion in Drug Development, 2000, 3(2), 293-213, and examples of suitable and preferred nucleoside analogues are provided by WO2007031091, which are hereby incorporated by reference.

“5-methylcytosine” means a cytosine modified with a methyl group attached to the 5′ position. A 5-methylcytosine is a modified nucleobase.

“2′-O-methoxyethyl” (also 2′-MOE and 2′-O(CH˜)˜—OCH3) refers to an O-methoxy-ethyl modification at the 2′ position of a furanose ring.

“2′-MOE nucleoside” (also 2′-O-methoxyethyl nucleoside) means a nucleoside comprising a 2′-MOE modified sugar moiety.

A “locked nucleic acid” or “LNA” is often referred to as inaccessible RNA, and is a modified RNA nucleobase. The ribose moiety of an LNA nucleobase is modified with an extra bridge connecting the 2′ oxygen and 4′ carbon. An LNA oligonucleotide offers substantially increased affinity for its complementary strand, compared to traditional DNA or RNA oligonucleotides. In some aspects bicyclic nucleoside analogues are LNA nucleotides, and these terms may therefore be used interchangeably, and in such embodiments, both are characterized by the presence of a linker group (such as a bridge) between C2′ and C4′ of the ribose sugar ring. When used in the present context, the terms “LNA unit”, “LNA monomer”, “LNA residue”, “locked nucleic acid unit”, “locked nucleic acid monomer” or “locked nucleic acid residue”, refer to a bicyclic nucleoside analogue. LNA units are described in inter alia WO 99/14226, WO 00/56746, WO 00/56748, WO 01/25248, WO 02/28875, WO 03/006475, WO2015071388, and WO 03/095467.

“Beta-D-Oxy LNA”, is a preferred LNA variant.

“Bicyclic nucleic acid” or “BNA” or “BNA nucleosides” mean nucleic acid monomers having a bridge connecting two carbon atoms between the 4′ and 2′ position of the nucleoside sugar unit, thereby forming a bicyclic sugar. Examples of such bicyclic sugar include, but are not limited to A) pt-L-methyleneoxy (4′-CH2-O-2′) LNA, (B) P-D-Methyleneoxy (4′-CH2-O-2′) LNA, (C) Ethyleneoxy (4′-(CH2)2-O-2′) LNA, (D) Aminooxy (4′-CH2-O—N(R)-2′) LNA and (E) Oxyamino (4′-CH2-N(R)-O-2′) LNA.

As used herein, LNA compounds include, but are not limited to, compounds having at least one bridge between the 4′ and the 2′ position of the sugar wherein each of the bridges independently comprises 1 or from 2 to 4 linked groups independently selected from ˜[C(R˜)(R2)]ₙ—, —C(R˜)═C(R2)-, —C(R˜)═N, —C(═NREM)-, —C(═O)—, —C(═S)—, —O—, —Si(Ri)q-, —S(═O)— and —N(R&)-; wherein: x is 0, 1, or 2; n is 1, 2, 3, or 4; each R& and R2 is, independently, H, a protecting group, hydroxyl, C>>C>>alkyl, substituted C>>(-CHz-) group connecting the 2′ oxygen atom and the 4′ carbon atom, for which the term methyleneoxy (4′-CH&-O-2′) LNA is used.

Furthermore, in the case of the bicyclic sugar moiety having an ethylene bridging group in this position, the ethyleneoxy (4′-CH&CH&-O-2′) LNA is used. n-L-methyleneoxy (4′-CH&-O-2′), an isomer of methyleneoxy (4′-CH&-O-2′) LNA is also encompassed within the definition of LNA, as used herein.

In some embodiments, the nucleoside unit is an LNA unit selected from the list of beta-D-oxy-LNA, alpha-Loxy-LNA, beta-D-amino-LNA, alpha-L-amino-LNA, beta-D-thio-LNA, alpha-L-thio-LNA, 5′-methyl-LNA, beta-D-ENA and alpha-L-ENA.

“cEt” or “constrained ethyl” means a bicyclic sugar moiety comprising a bridge connecting the 4′-carbon and the 2′-carbon, wherein the bridge has the formula: 4′-CH(CHq)-O-2′.

“Constrained ethyl nucleoside” (also cEt nucleoside) means a nucleoside comprising a bicyclic sugar moiety comprising a 4′-CH(CH3)-O-2′ bridge. cEt and some of its properties are described in Pallan et al. Chem Commun (Camb). 2012 Aug. 25; 48(66): 8195-8197.

“Tricyclo (tc)-DNA” belongs to the class of conformationally constrained DNA analogs that show enhanced binding properties to DNA and RNA. Structure and method of production may be seen in Renneberg et al. Nucleic Acids Res. 2002 Jul. 1; 30(13): 2751-2757.

“2′-fluoro”, as referred to herein is a nucleoside comprising a fluoro group at the 2′ position of the sugar ring. 2′-fluorinated nucleotides are described in Peng et al. J Fluor Chem. 2008 September; 129(9): 743-766.

“2′-O-methyl”, as referred to herein, is a nucleoside comprising a sugar comprising an —OCH3 group at the 2′ position of the sugar ring.

“Conformationally Restricted Nucleosides (CRN)” and methods for their synthesis, as referred to herein, are described in WO2013036868, which is hereby incorporated by reference. CRN are sugar-modified nucleosides, in which, similar to LNA, a chemical bridge connects the C2′ and C4′ carbons of the ribose. However, in a CRN, the C2′-C4′ bridge is one carbon longer than in an LNA molecule. The chemical bridge in the ribose of a CRN locks the ribose in a fixed position, which in turn restricts the flexibility of the nucleobase and phosphate group. CRN substitution within an RNA- or DNA-based oligonucleotide has the advantages of increased hybridization affinity and enhanced resistance to nuclease degradation.

“Unlocked Nucleic Acid” or “UNA”, is as referred to herein unlocked nucleic acid typically where the C2-C3 C—C bond of the ribose has been removed, forming an unlocked “sugar” residue (see Fluiter et al., Mol. Biosyst., 2009, 10, 1039, hereby incorporated by reference, and Snead et al. Molecular Therapy—Nucleic Acids (2013) 2, e103;).

“Target region” means a portion of a target nucleic acid to which one or more antisense compounds is targeted.

“Targeted delivery” as used herein means delivery, wherein the antisense oligonucleotide has either been formulated in a way that will facilitate efficient delivery in specific tissues or cells, or wherein the antisense oligonucleotide in other ways has been for example modified to comprise a targeting moiety, or in other way has been modified in order to facilitate uptake in specific target cells.

The antisense oligonucleotides of the invention are designed to target microRNA-134 (miR-134) Specific antisense oligonucleotides have been designed to target regions of miR-134 having the mature sequence 5′ UGUGACUGGUUGACCAGAGGGG 3′ (SEQ ID NO: 1) (miRBase acc #MIMAT0000447).

The above reference to “miRBase” are according to miRBase release 22.1.

The term “miR-134 related neurological disease” as used herein means diseases where disease pathology is linked with upregulation of miR-134 activity, or where downregulation of miR-134 activity will be beneficial for treatment of the disease.

### Compounds

The compounds of the present invention are LNA/DNA mixmer antisense oligonucleotides targeting miR-134 (antimiR-134 compounds) comprising a sequence of 17-19 nucleotides in length, such as 18-19 nucleotides in length, wherein the antisense oligonucleotides are complementary to one or more of SEQ ID NO: 1-3. The antimiR-134 compounds of the invention are 17, 18 or 19 nucleotides in length, have at least two terminal LNA nucleotide analogues at the 3′-end, they comprise between 40% and 70% LNA, such as between 50% and 70% LNA, and do not comprise a consecutive stretch of more than two DNA nucleotides. Furthermore, the antimiR-134 compounds of the invention have at least one phosphorothioate internucleotide linkage. According to an aspect, the invention concerns an antimiR-134 oligonucleotide complementary to miR-134 consisting of a sequence of 18-19 nucleobases in length that is a mixmer comprising from seven to 14, such as from ten to 14 affinity-enhancing nucleotide analogues and does not contain a stretch of more than three contiguous DNA nucleotides, and wherein the antisense oligonucleotide comprises 1 to 18 phosphorothioate internucleoside linkages, and wherein the antisense oligonucleotide is complementary to SEQ ID NO: 2.

Specific compounds of the invention are disclosed in Table 1 as SEQ ID NO's: 5-21 or 23-60. In some embodiments, the compounds of the invention, such as SEQ ID NO's: 5-21 or 23-60 comprise other nucleotide analogues than LNA. In some embodiments, some of the DNA nucleotides in SEQ ID NO's: 5-21 or 23-60 are replaced with other affinity enhancing nucleotide analogues than LNA, such as in a non-limiting example anyone of tricyclo-DNA, 2′-Fluoro, 2′-O-methyl, 2′methoxyethyl (2′MOE), 2′ cyclic ethyl (cET), UNA, 2′fluoro and Conformationally Restricted Nucleoside (CRN). In some embodiments the compounds SEQ ID NO's: 5-21 or 23-60 have a complete phosphorothicate backbone, i.e. all internucleoside linkages are phosphorothicate linkages. In a preferred embodiment, in the compounds SEQ ID NO's: 5-21 or 23-60 as shown in Table 1 all internucleoside bonds are phosphorothicate bonds, all LNAs are beta-D-oxy LNA and LNA cytosines (C) all are 5-methylcytosine.

TABLE 1
Antisense oligonucleotide compounds
complementary to miR-134

| | | length | # | % |
|---|---|---|---|---|
| Seq id # | Sequence (5′-3′) | (nt) | LNA | LNA |
| 5 | CtcTGgTcaACcaGTcAC | 18 | 11 | 61 |
| 6 | CtcTgGTcaACcAgTcAC | 18 | 11 | 61 |
| 7 | CTctGgTCaACcaGtCAC | 18 | 11 | 61 |
| 8 | CtctgGTcaAcCAgTcAC | 18 | 10 | 55 |
| 9 | CTctGGTcaAcCaGTcAC | 18 | 11 | 61 |
| 10 | CtctgGTcaACcAgTcAC | 18 | 10 | 55 |
| 11 | CTCTGgtCaaCcAGTcAC | 18 | 11 | 61 |
| 12 | CTCtgGTcaAccAGTcAC | 18 | 11 | 61 |
| 13 | CTCtgGtcAAccAGtcAC | 18 | 10 | 55 |
| 14 | CTctGgTcAAcCAGtcAC | 18 | 11 | 61 |
| 15 | CTcTgGtCaaCCAgtCAC | 18 | 11 | 61 |
| 16 | CTctGGtcAAcCagTCAC | 18 | 11 | 61 |
| 17 | CCtcTGgtCaaCcAGtCAC | 19 | 11 | 58 |
| 18 | CCtcTgGTcaACcaGTcAC | 19 | 11 | 58 |
| 19 | CCTctGgTcAAccAGtcAC | 19 | 11 | 58 |
| 20 | CTCtgGtcAAccAGtcAC | 18 | 10 | 55 |
| 21 | CCTctGgTcAAccAGtcAC | 19 | 11 | 58 |
| 22 | TgGtcAAccAgTcAC | 15 | 8 | 53 |
| (comparator) | | | | |
| 23 | TCtgGTcaAccaGTcACA | 18 | 10 | 55 |
| 24 | TCtgGTcaAccAgTcACA | 18 | 10 | 55 |
| 25 | TCtgGTcaAccAGtcACA | 18 | 10 | 55 |
| 26 | TCtgGTcaAccAGTcACA | 18 | 11 | 61 |
| 27 | CTctgGtcaAcCaGTcACA | 19 | 10 | 53 |
| 28 | CTctGgTcAAccAgTcACA | 19 | 11 | 58 |
| 29 | CTctGgTcAAccAGtcACA | 19 | 11 | 58 |
| 30 | CTctGgTcAAccAGTcACA | 19 | 12 | 63 |
| 31 | TCtgGTcaAccaGTcAC | 17 | 9 | 53 |
| 32 | TCtgGTcaAccAgTcAC | 17 | 9 | 53 |
| 33 | TCtgGTcaAccAGtcAC | 17 | 9 | 53 |
| 34 | TCtgGTcaAccAGTcAC | 17 | 10 | 59 |
| 35 | CCtctGgTcAAccaGTcAC | 19 | 10 | 53 |
| 36 | CCtctGgTcAAccAgTcAC | 19 | 10 | 53 |
| 37 | CCtctGgTcAAccAGtcAC | 19 | 10 | 53 |
| 38 | CTctgGtcaAcCagtCA | 17 | 7 | 41 |
| 39 | CTctgGtcaAcCagTCA | 17 | 8 | 47 |
| 40 | CTctgGtcaAcCaGtCA | 17 | 8 | 47 |
| 41 | CTctgGtcaAcCaGTCA | 17 | 9 | 53 |
| 42 | CTctGgTcAAccAgtCA | 17 | 9 | 53 |
| 43 | CTctGgTcAAccAgTCA | 17 | 10 | 59 |
| 44 | CTctGgTcAAccAGtCA | 17 | 10 | 59 |
| 45 | CCTctGgTcAAcCagtCA | 18 | 10 | 55 |
| 46 | CCtcTggTcaACcagTCA | 18 | 9 | 50 |
| 47 | CCtctGgTcAAccaGtCA | 18 | 9 | 50 |
| 48 | CCtctGgTcAAccaGTCA | 18 | 10 | 55 |
| 49 | CCtctGgTcAAccAgtCA | 18 | 9 | 50 |
| 50 | CCtctGgTcAAccAgTCA | 18 | 10 | 55 |
| 51 | CCtctGgTcAAccAGtCA | 18 | 10 | 55 |
| 52 | CCTctGgTcAAccAgTcAC | 19 | 11 | 58 |
| 53 | CCTctGgTcAAccaGTcAC | 19 | 11 | 58 |
| 54 | CTCtgGTcaAccAgTcAC | 18 | 10 | 55 |
| 55 | CTCtgGTcaAccAGtcAC | 18 | 10 | 55 |
| 56 | CTCtgGTcaAccaGTcAC | 18 | 10 | 55 |
| 57 | CTcTgGTcaACcAGtcAC | 18 | 11 | 61 |
| 58 | CTcTgGTcaACcAGTcAC | 18 | 12 | 67 |
| 59 | CTcTgGTcaACcaGTcAC | 18 | 11 | 61 |
| 60 | CCtcTgGTcaACcAgTcAC | 19 | 11 | 58 |

In Table 1, uppercase letters indicate LNA nucleotides, lowercase letters are DNA nucleotides, LNA cytosine is 5-methylcytosine, all internucleoside bonds are phosphorothioate bonds, and LNA is beta-D-oxy LNA.

The control (comparator) oligonucleotide SEQ ID NO: 22 is identical to SEQ ID NO: 8 of WO19219723.

According to one aspect, the present invention concerns a miR-134 inhibitory composition comprising the antisense oligonucleotides complementary to miR-134 according to the invention and/or embodiments.

According to another aspect the invention concerns a pharmaceutical composition comprising an effective dosage of the antisense oligonucleotide complementary to miR-134 according to the invention and/or embodiments and a pharmaceutically acceptable carrier.

According to another aspect the invention concerns a pharmaceutical composition comprising an effective dosage of the antisense oligonucleotide complementary to miR-134 according to the invention and/or embodiments, wherein said antisense oligonucleotide complementary to miR-134 is the sole active pharmaceutical ingredient.

According to another aspect the invention concerns a method of treatment of the diseases according to the invention and/or embodiments by use of the antisense oligonucleotides according to the invention and/or embodiments or the composition according to the invention and/or embodiments. In some aspects, the invention relates to the compounds of the invention, i.e. any one of Seq ID NO's 5-21 or 23-60, such as in preferred embodiment, anyone of SEQ ID NO's 7, 8, 10, 12, 19, 24, 26-37, 45-56, 59 or 60 for use as a medicament.

According to another aspect the invention concerns a method of diagnosing a disease according to the invention and/or embodiments by use of the antisense oligonucleotide complementary to miR-134 according to the invention and/or embodiments or the composition according to the invention and/or embodiments.

### Compositions and Uses

The compounds of the invention are for use in the compositions, such as in the pharmaceutical compositions of the invention, and for the use as medicaments, and for treatment, alleviation, amelioration, pre-emptive treatment, or prophylaxis of the diseases disclosed herein, such as neurological disorders, including epilepsy.

The compounds of the invention are in some embodiments comprised in compositions, such as pharmaceutical compositions for treatment of miR-134 related diseases, which are diseases where modulation of miR-134 activity is beneficial for treatment, prophylaxis, alleviation or amelioration of the disease or disease parameters. In some embodiments, the treatment, prophylaxis, alleviation or amelioration is curative. In some embodiments, the treatment, prophylaxis, alleviation or amelioration is disease modifying. In some embodiments, the treatment, prophylaxis, alleviation or amelioration is preventive.

Diseases that may be treated, alleviated, ameliorated, pre-emptively treated or prophylactically treated by the compounds and compositions include in non-limiting example diseases of the central nervous system (CNS) or peripheral nervous system (PNS), including neurological, such as neurodegenerative disorders, neurodevelopmental disorders, genetic disorders, genetic neurodevelopmental disorders, or psychiatric diseases. Specifically the compounds and compositions of the invention may be used for treatment, alleviation, amelioration, pre-emptive treatment or prophylaxis of epilepsy, such as of drug resistant epilepsy, of seizures in epilepsy, of spontaneous seizures in epilepsy, of therapy resistant seizures, of focal epilepsy, preferably wherein said focal epilepsy is focused in the frontal lobe, the parietal lobe, the occipital lobe or the temporal lobe. In some embodiments, the epilepsy is a generalised epilepsy, preferably selected among absences, myoclonic seizures, tonic-clonic seizures, tonic seizures, atonic seizures, clonic seizures and spasms. In some embodiments, the epilepsy is status epilepticus. In some embodiments, the compounds and compositions of the invention are for treatment, alleviation, amelioration, pre-emptive treatment or prophylaxis of epilepsy, such as of epilepsy selected among autosomal dominant nocturnal frontal lobe epilepsy, continuous spike-and-waves during slow sleep, Dravet syndrome, epilepsy developed after apoplexy, epileptic encephalopathy, Gelastic epilepsy, absences, benign neonatal seizures, Jeavons syndrome, Juvenile myoclonic epilepsy, Landau-Kleffner Syndrom, Lennox-Gastaut syndrome, Mesial temporal lobe epilepsy, myoclonic astatic epilepsy, Ohtahara Syndrom, Panayiotopoulos syndrome, PCDH19 syndrom, benign childhood epilepsy with centrotemporal spikes, Sturge-Weber syndrome, symptomatic focal epilepsy, transient epileptic amnesia and West syndrome. In some embodiments, the compounds and compositions of the invention are for treatment, alleviation, amelioration, pre-emptive treatment or prophylaxis of epilepsy wherein said epilepsy is present together with a comorbidity selected among a psychiatric disorder, a cognitive disorder, a sleep disorder, a cardiovascular disorder, a respiratory disorder, an inflammatory disorder, anxiety, pain, cognitive impairment, depression, dementia, headache, migraine, heart disease, ulcers, peptic ulcers, arthritis and osteoporosis.

In some embodiments, the compounds and compositions of the invention are for treatment, alleviation, amelioration, pre-emptive treatment or prophylaxis of neuronal damage, such as hippocampal damage. In some embodiments, the compounds and compositions of the invention are useful for treatment, alleviation, amelioration, pre-emptive treatment or prophylaxis of oxidative stress, inflammation and apoptosis. In some embodiments, the compounds and compositions of the invention are for treatment, alleviation, amelioration, pre-emptive treatment or prophylaxis of intracerebral hemorrhage-induced brain injury, ischemic stroke, haemorrhagic stroke or stroke. In some embodiments, the compounds and compositions of the invention are for treatment, alleviation, amelioration, pre-emptive treatment or prophylaxis of an autoimmune disease, a memory disorder, hippocampal sclerosis, Parkinsons Disease, a demyelinating disease, multiple sclerosis, spinal cord injury, acute spinal cord injury, amyotrophic lateral sclerosis, progressive bulbar palsy, progressive muscular atrophy, primary lateral sclerosis, ataxia, bell's palsy, a hereditary neurological disease, Charcot-Marie-Tooth, a headache, Horton's headache, migraine, pick's disease, progressive supranuclear palsy, multi-system degeneration, motor neuron diseases, Huntington's disease, prion disease, Creutzfeldt-Jakob disease, corticobasal degeneration, aphasia, primary progressive aphasia, a movement disorder or symptoms or effects thereof.

In some embodiments, the compounds and compositions of the invention are for treatment, alleviation, amelioration, pre-emptive treatment or prophylaxis of dementia.

In some embodiments, the compounds and compositions of the invention are for treatment, alleviation, amelioration, pre-emptive treatment or prophylaxis of dementia is selected among Alzheimer disease, vascular dementia, frontotemporal dementia and Lewy bodies dementia. In some embodiments, the compounds and compositions of the invention are for treatment, alleviation, amelioration, pre-emptive treatment or prophylaxis of pain.

The compounds of the invention may also be used for treatment of certain psychiatric diseases, in particular those where modulation of miR-134 activity is beneficial.

In some embodiments, the compounds and compositions of the invention are for treatment, alleviation, amelioration, pre-emptive treatment or prophylaxis of a psychiatric disease wherein modulation of miR-134 is beneficial.

In some embodiments, the compounds and compositions of the invention are for treatment, alleviation, amelioration, pre-emptive treatment or prophylaxis of autism, or a mood disorder, depressive disorder, schizophrenia, bipolar disorder, attention deficit hyperactivity disorder, anxiety or Tourette.

In some embodiments, the compounds and compositions of the invention are for treatment, alleviation, amelioration, pre-emptive treatment or prophylaxis of major depressive disorder. In some embodiments, the compounds and compositions of the invention are for treatment, alleviation, amelioration, pre-emptive treatment or prophylaxis of a memory disorder, wherein modulation of miR-134 expression or activity is beneficial.

miR-134 is implicated in the pathogenesis of various cancers, and the antimiR-134 compounds of the invention are therefore also for use in methods of treating or preventing cancer.

In some embodiments, the compounds and compositions of the invention are for treatment, alleviation, amelioration, pre-emptive treatment or prophylaxis of a cancer.

In some embodiments, the compounds and compositions of the invention are for treatment, alleviation, amelioration, pre-emptive treatment or prophylaxis of a cancer in the nerve system, preferably glioma.

In some embodiments, the compounds and compositions of the invention are for treatment, alleviation, amelioration, pre-emptive treatment or prophylaxis of cancer selected from the group of lung tumors, non-small cell lung cancer, glioma, head and neck squamous cell carcinoma, pancreatic cancer, colon cancer, prostate cancer, melanoma, uveal melanoma, oral squamous cell carcinoma or squamous cell carcinoma of the tongue.

In some embodiments, the compounds and compositions of the invention are for treatment, alleviation, amelioration, pre-emptive treatment or prophylaxis of anyone of Prader-Willis Syndrome, Anglemans Syndrome, a cardiovascular disorder, atherosclerosis, and pulmonary disease.

In some embodiments, the compounds and compositions of the invention are for treatment, alleviation, amelioration, pre-emptive treatment or prophylaxis of an infection.

In some embodiments, the compounds and compositions of the invention are for treatment, alleviation, amelioration, pre-emptive treatment or prophylaxis of and infection selected among, sepsis, meningitis and encephalitis.

In some embodiments, the compounds and compositions of the invention are for treatment, alleviation, amelioration, pre-emptive treatment or prophylaxis of

In some embodiments, the compounds and compositions of the invention are for treatment, alleviation, amelioration, pre-emptive treatment or prophylaxis of a genetic disorder, preferably neurofibromatosis.

In some embodiments, the antimiR-134 compounds may advantageously be used together with other therapies for a certain disease to be treated by the antimiR-134 composition.

Thereby, the antisense oligonucleotides complementary to miR-134 of the invention is for use in combination with one or more other therapies. In some embodiments the antisense oligonucleotide complementary to miR-134 of the invention is for use in combination with one or more other therapies for the diseases mentioned in the embodiments, such as for treatment of neurological and psychiatric disorders. In some embodiments, said other therapy is an anti miR-27b antisense oligonucleotide. In some embodiments, said other therapy is said therapy is an adenosine kinase inhibitor. In some embodiments, said other therapy induces the Nrf-2/ARE pathway in a mammal, such as in a human. In some embodiments, the antimiR-134 compositions are to be used in combination with one or more of an anti miR27b antisense oligonucleotide, an anti adenosine kinase antisense oligonucleotide and a therapy inducing the Nrf-2/ARE pathway.

In some embodiments, the antisense oligonucleotides of the invention are to be used in compositions where they are the sole active ingredient, and in some embodiments, they are for use in compositions comprising other active pharmaceutical ingredients.

The invention provides pharmaceutical compositions comprising the antimiR-134 compounds of the invention further comprising a pharmaceutically acceptable carrier.

In some embodiments, the pharmaceutical compositions of the invention comprises the antisense oligonucleotide complementary to miR-134 as the sole active pharmaceutical ingredient. In some embodiments, one or more active pharmaceutical ingredients are present in the pharmaceutical compositions of the invention.

### Dosages

The expression “effective dosage” denotes the dose of a drug that will achieve the desired effect. In the context of the present invention, the desired effect is lowering of the activity of miR-134. Lowering of the activity of miR-134 can be measured by either measuring the level of miR-134, for example when using oligonucleotides which result in degradation of miR-134 or miR-134 precursors, or may be measured by measuring the derepression of microRNA-134 targets (such as mRNAs which comprise a miR-134 binding site and whose expression is regulated by miR-134 (miR-134 target mRNAs)). miR-134 inhibition may therefore be measured directly or indirectly via secondary indicators of miR-134 activity.

The compounds of the invention are for use in effective dosages, and the compositions comprise effective dosages of the compounds of the invention.

In some embodiments, the dosage of the compound administered at each dosing, such as unit dose, is within the range of 0.0001 mg/kg-25 mg/kg.

In some embodiments, the effective dose is a dose that is sufficient to down-regulate miR-134 or the activity thereof, to a significant level over the time period between successive administration dosages, such as a level which is a therapeutic benefit to the subject.

The pharmaceutical compositions of the invention may in some embodiments be made for administration to provide for an initial dosage build up phase, which may, depending on the disease pathology, be followed by a maintenance dosage scheme for the purpose of maintaining a concentration of the compound in the subject, such as in a target tissue of the subject, which will be effective in the treatment of the disease. The effectiveness of the dosages may in example be measured by observation of a disease parameter indicative of the state of the disease, or may depending on the target tissue, be measurable by observation of various tissue parameters, such as activity of a miR-134 target RNA, or in alternative example on a measurable disease state dependent parameter in plasma.

### Drug Delivery

Various delivery systems are known and can be used to administer a therapeutic of the invention. Methods of administration includes but are not limited to subcutaneous administration, intravenous administration, parenteral administration, nasal administration, pulmonary administration, rectal administration, vaginal administration, intrauterine administration, Intraurethral administration, administration to the eye, administration to the ear, cutaneous administration, intradermal administration, intramuscular administration, intraperitoneal administration, epidural administration, intraventricular administration, intracerebral, intrathecal administration or oral administration or administration directly into the brain or cerebrospinal fluid. The compositions may be administered by any convenient route, for example by infusion or bolus injection, by absorption through epithelial or mucocutaneous tissue (e.g., oral mucosa, rectal and intestinal mucosa, etc.) and may be administered together with or without other biologically active agents. Administration can be systemic or local. In addition, it may be desirable to administer the compositions of the invention into the central nervous system by any suitable route, including intraventricular and intrathecal administration. Intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. Preferably, the therapeutic is delivered to the CNS or PNS.

Delivery means include inhaled delivery, intramuscular delivery directly into a muscle by syringe or mini osmotic pump, intraperitoneal administration directly administered to the peritoneum by syringe or mini osmotic pump, subcutaneous administration directly administered below the skin by syringe, intraventricular administration direct administration to the ventricles in the brain, by injection or using small catheter attached to an osmotic pump. Further, an implant can be prepared (e.g. small silicon implant) that will be placed in a muscles or directly onto the spinal cord. It may be desirable to administer the compositions of the invention locally to the area in need of treatment; this may be achieved for example and not by way of limitation, by topical application, by injection, by means of a catheter, by means of a suppository, or by means of an implant, said implant may be of a porous, non-porous, or gelatinous material, including membranes, such as sialastic membranes, or fibers.

### Pharmaceutical Compositions

The present invention also provides pharmaceutical compositions. Such compositions may comprise a therapeutically effective amount of the therapeutic, and a pharmaceutically acceptable carrier. The term “pharmaceutically acceptable” may be defined as approved by a regulatory agency. The regulatory agency may for example be the European Medicines Agency, a Federal or a state government or listed in the U.S. Pharmacopeia or other generally recognized pharmacopeia for use in animals, and more particularly in humans. The term “therapeutically effective amount” may be defined as an amount of therapeutic which results in a clinically significant inhibition, amelioration or reversal of development or occurrence of a disorder or disease. The term “carrier” may refer to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water may be a preferred carrier when the pharmaceutical composition is administered intravenously. Saline solutions and aqueous dextrose and glycerol solutions may also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, may also contain wetting or emulsifying agents, or pH buffering agents. These compositions may take the form of solutions, suspensions, emulsion, tablets, pills, capsules, powders, sustained-release formulations and the like. The composition may be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. Such compositions may contain a therapeutically effective amount of the therapeutic, preferably in purified form, together with a suitable amount of carrier so as to provide the form for proper administration to the patient. The formulation may suit the mode of administration. Compositions for intravenous administration may be solutions in sterile isotonic aqueous buffer. Where necessary, the composition may also include a solubilizing agent and a local anaesthetic such as lignocaine to ease pain at the site of the injection. The ingredients may be supplied either separately or mixed together in unit dosage form, for example, as a dry lyophilized powder or water free concentrate in a hermetically sealed container such as an ampoule or sachette indicating the quantity of active agent. Where the composition is to be administered by infusion, it may be dispensed with an infusion bottle containing sterile pharmaceutical grade water or saline. Where the composition is administered by injection, an ampoule of sterile water for injection or saline may be provided so that the ingredients may be mixed prior to administration.

### BRIEF DESCRIPTION OF THE DRAWINGS

FIG. 1 shows the levels of repression of Renilla signal normalized to Firefly as percent of empty vector, n,N=2-3,4-6, mean±SEM. The most potent anti-sense oligonucleotides are SEQ ID No. 7, 8, 10, 12 and 19.

FIG. 2 shows the dose-response curves and the IC₅₀ values of the five miR-134 antisense oligonucleotides. Dose-response curves and IC₅₀ values, n(N,=1, 2, both technical replicates are shown, 3-parameter non-linear curve fit.

FIG. 3 shows IC50 values measured by derepression of Renilla luciferase activity in U-87 mg cells, n,N=2,4-6, all biological replicates are depicted. IC50 curves were fitted, and potency calculated using least squares regression with log(inhibitor) vs. a three-parameter response.

FIG. 4 shows increase in miR-134 direct and downstream target mRNAs after transfection of antimiR-134 oligonucleotides into PC-12 Adh cells; n,N=3,6; mean±SEM, all technical replicates are depicted. qPCR results were analysed using the ΔΔCt method using a scrambled oligonucleotide as normalization.

FIG. 5 shows increase in miR-134 direct target protein LIMK1, after transfection of antimiR-134 antisense oligonucleotides into PC-12 Adh cells; n=2; mean±SEM, all biological replicates are depicted. Western blotting results were analysed by densiometry using a GAPDH as normalisation and are expressed in percent of scrambled control.

FIG. 6 shows the levels of Renilla luciferase signal derepression caused by Seq ID Nos 23-60, normalized to Firefly. n(N)=2-3 (4-6), mean±SEM. All technical replicates are shown.

FIG. 7 shows IC50 values of SEQ ID NO 19, 36, 52, 53, and 54 measured by derepression of Renilla luciferase activity in PC-12 Adh cells, n,N=3, 4-6. All biological replicates are shown. IC50 curves were fitted, and potency calculated using least squares regression with log(inhibitor) vs. a three-parameter response.

### EXAMPLES

### Example 1: Cell Culture

The adherent rat pheochromocytoma cell line PC-12 Adh (ECACC no. 88022401) was purchased from ATCC (ATCC cat. no. CRL-1721.1™) and grown in Corning® CellBIND® Surface cell culture flasks (Sigma-Aldrich cat. no. CLS3290) in Ham's F-12K (Kaighn's) medium (ThermoFischer Scientific cat. no. 21127022) supplemented with 2.5% heat-inactivated fetal bovine serum (Sigma-Aldrich cat. no F4135-500 ml), 15% heat-inactivated horse serum (Sigma-Aldrich cat. no. H1385-500 ml and 1% penicillin/streptomycin (Sigma-Aldrich cat. no. P4333-100 ml). The cells were kept in in a humidified 5% CO2 incubator at 37° C. and passaged twice a week.

### Example 2: Luciferase Reporter Assays in Cultureds

A simple and very sensitive approach involves construction of a miRNA reporter plasmid that carries a single perfect match miRNA binding site in the 3′ UTR of a reporter gene, such as luciferase. This method has been extensively used in cultured cells to validate miRNA inhibition and also to compare the potency of different antimiR designs.

The miR-134 reporter was generated by cloning annealed oligonucleotides corresponding to single perfect-match target site for human miR-134 into the 3′ UTR of the Renilla luciferase gene in the dual-luciferase psiCHECK2 plasmid (Promega).

For luciferase assays, PC-12 Adh cells were seeded in 96-well Corning® CellBIND® Surface cell culture microwell plates (Sigma-Aldrich cat. no. CLS3330) at a density of 25,000 cells per well the day before transfection. The cells were transfected using lipofectamine 2000 (ThermoFischer Scientific cat. no. 11668-019) at a final concentration of 0.5 L/well in Opti-MEM™ | Reduced Serum Medium, GlutaMAX™ Supplement (ThermoFischer Scientific cat. no. 51985026). Since miR-134 is downregulated in a wide range of cancers (Pan et al, Mol Ther Nucleic Acids. 2017 Mar. 17; 6: 140-149.) it was challenging to find a cell line expressing sufficient levels of miR-134. To overcome this a miR-134 mimic was co-transfected into the cells. Thus, a final concentration of 2.5 nM of miRCURY LNA miRNA Mimic, hsa-miR-134-5p (Qiagen cat no. 339173) was added to the transfection reaction. A library of 17 antisense oligonucleotides was screened using the luciferase reporter assays by co-transfecting each antimiR-134 with the luciferase reporter plasmid and the miR-134 mimic in final concentrations of 0.2 nM, 1 nM, 5 nM. A scrambled sequence oligonucleotide, a vector containing no miRNA match site and a mock transfection were included as controls. Furthermore, a previously published antimiR-134 oligonucleotide (SEQ ID NO:8 of WO19219723) was used as comparator. All samples were run in technical duplicates. After four hours the cells were washed in Opti-MEM™ medium and fresh complete cell culture medium was added to the wells.

24 hours after transfection the luciferase assay was conducted using Dual-Glo® Luciferase Assay System (Promega cat. no. E2920) as per manufacturer's instructions. The amount of luminescence was determined on a plate reader (VarioSkan Lux, ThermoFischer Scientific) after 30 minutes incubation of reagents in the plates.

The results were analysed by subtraction of background luminescence and then normalizing the Renilla luciferase activity by the Firefly luciferase activity. The average of the two technical duplicates were then normalized to empty vector and expressed as percentage. The results were visualized in Graphpad Prism (version 9.0.2, GraphPad Software).

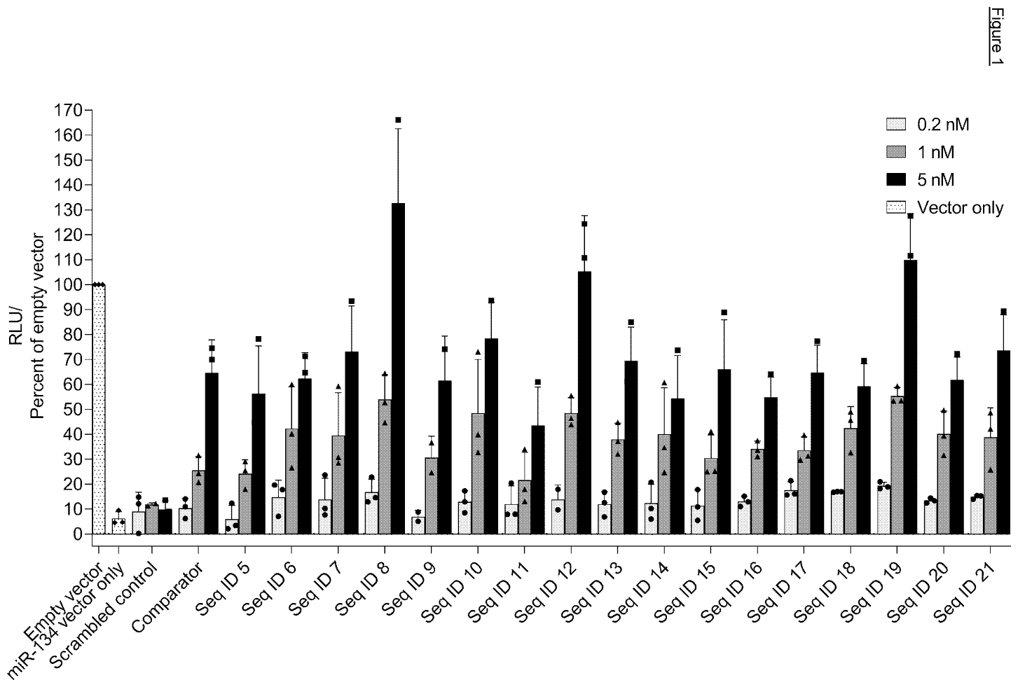

The levels of derepression of Renilla luciferase activity normalized to Firefly luciferase activity in percentage of empty vector for all 17 antisense oligonucleotides are shown in FIG. 1.

From the full library of antimiR-134 oligonucleotides the five most potent antimiR 134 molecules were chosen for further analyses and IC₅₀ determinations.

### Example 3: Determination of IC50 for antimiR-134 Oligonucleotides in Cultured Cell Lines

To determine the potency of antisense oligonucleotides in inhibiting miR-134, IC50 determinations were conducted. The luciferase assays were carried out as described in example 2. The cells were transfected using a wide range of antimiR-134 concentrations ranging from 80 nM in 2-fold dilutions to 0.0049 nM. The Renilla luciferase was normalized to firefly luciferase activity and plotted against log(M) in Graphpad Prism (version 9.0.2, GraphPad Software). The dose-response curves were fitted using 3-parameter non-linear fit and IC50 values calculated in nM.

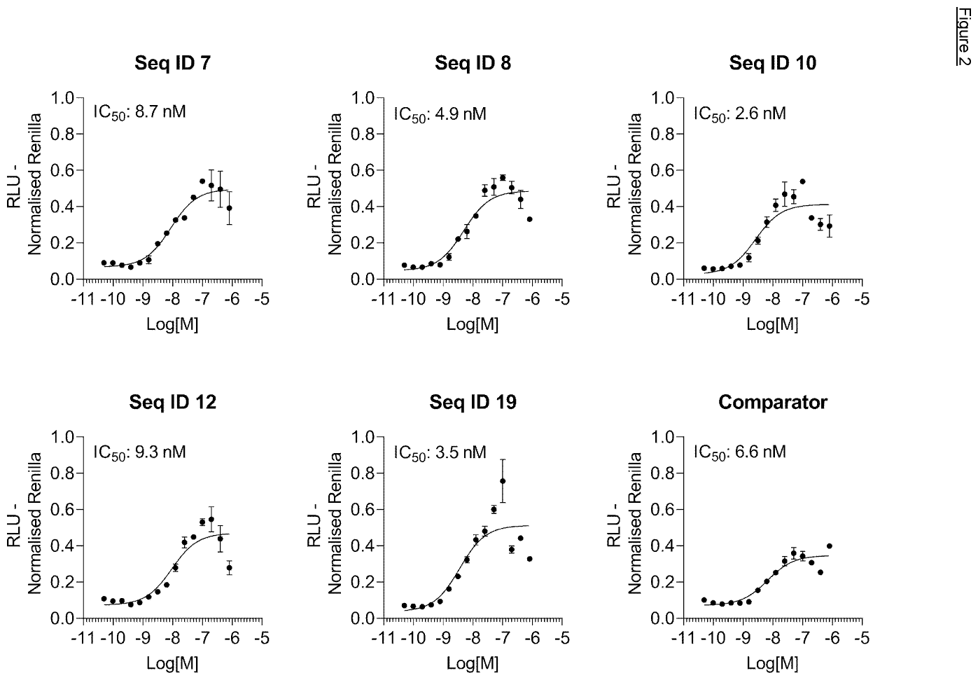

FIG. 2 shows the dose-response curves and the IC50 values of five antimiR-134 oligonucleotides.

### Example 4: IC50 Determination in Cultured U-87 Mg Cells

The IC50 curves in U-87 Mg cell lines were done as in the PC-12 Adh cells, except that the amount of Lipofectamine2000 was 0.4 μL per well and the transfections were done in 96-well Costar black plates (cat. no: 3603, Corning World, Corning, NY, USA).

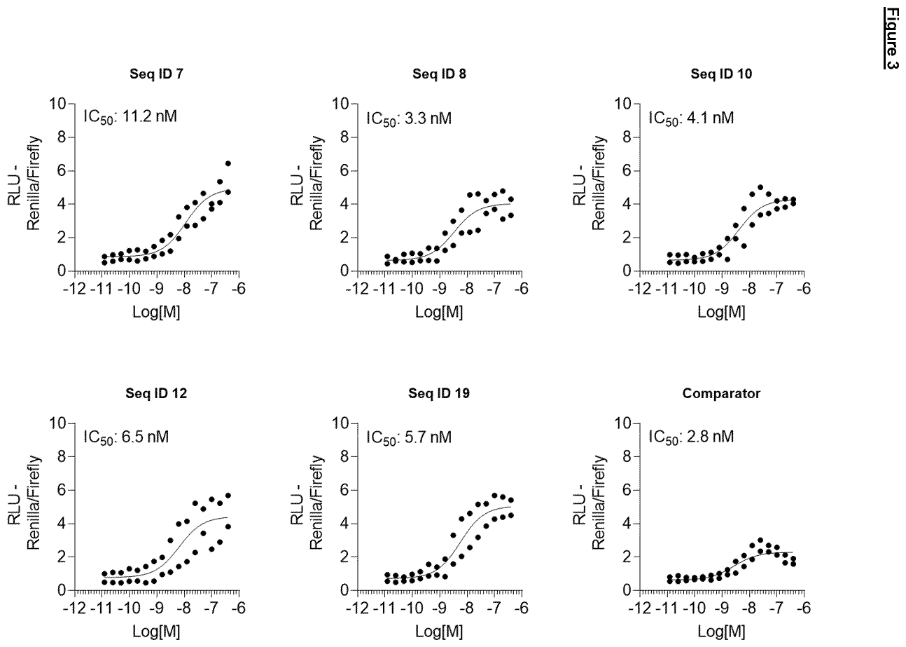

FIG. 3 shows the dose-response curves and the IC50 values of the five miR-134 antimiR oligonucleotides in U-87 Mg cells.

### Example 5: miR-134 Target mRNA Derepression in Cultured PC-12 Adh Cell Line

As miRNAs negatively regulate levels of their target mRNAs, the functional effects of miR-134 inhibition by antimiR oligonucleotides can be measured by a subsequent upregulation of target mRNAs. The principal targets of miR-134 are Limk1 and Serpine1. Upregulation in these two markers signify a functional effect of miR-134 inhibition.

The PC-12 Adh cells were transfected as described in the above examples with the exception that the cells were seeded in 12-well CellBind plates (cat. no: CLS3336, Corning World, Corning, NY, USA) at 3×105 cells/well, using 6 μL Lipofectamine2000 per well and no luciferase reporter was used. A FAM-labelled oligonucleotide was transfected in a separate well to confirm transfection efficiency by examination by direct microscopy. Forty-eight hours after transfection, RNA extraction was conducted using the miRNeasy mini kit (cat. no: 217004, Qiagen, Hilden, Germany) as per manufacturer's instructions. The RNA was stored at −80° C. until further analysis. Reverse transcription was conducted using Superscript IV reverse transcriptase (cat. no: 18090010, Thermo Fischer Scientific, Waltham, MA, USA) as per manufacturer's instructions, including gDNA removal by ezDNase™ (cat. no: 11766051, Thermo Fischer Scientific, Waltham, MA, USA) and using a random hexamer primer (cat. no: SO142, Thermo Fischer Scientific, Waltham, MA, USA). The qPCR was done on a QuantStudio 6 Flex (Applied Biosystems, Waltham, MA, USA) using Taqman assays (Table 2) synthesized by Integrated DNA Technologies (Newark, NJ, USA) and TaqMan™ Universal Master Mix II, no UNG (cat. no: 4440040, Thermo Fischer Scientific, Waltham, MA, USA) as per manufacturer's instructions. All qPCR assays were designed to be exon-spanning and specificity was confirmed by blast of the primers and the efficiency of primers was tested using a five-fold dilution series. Hprt1 was used as a house-keeping gene. All qPCR results were analysed using the ΔΔCt method (Livak K J, Schmittgen T D. Analysis of Relative Gene Expression Data Using Real-Time Quantitative PCR and the 2-ΔΔCT Method. Methods. 2001; 25(4):402-408) using a scrambled oligonucleotide for normalisation.

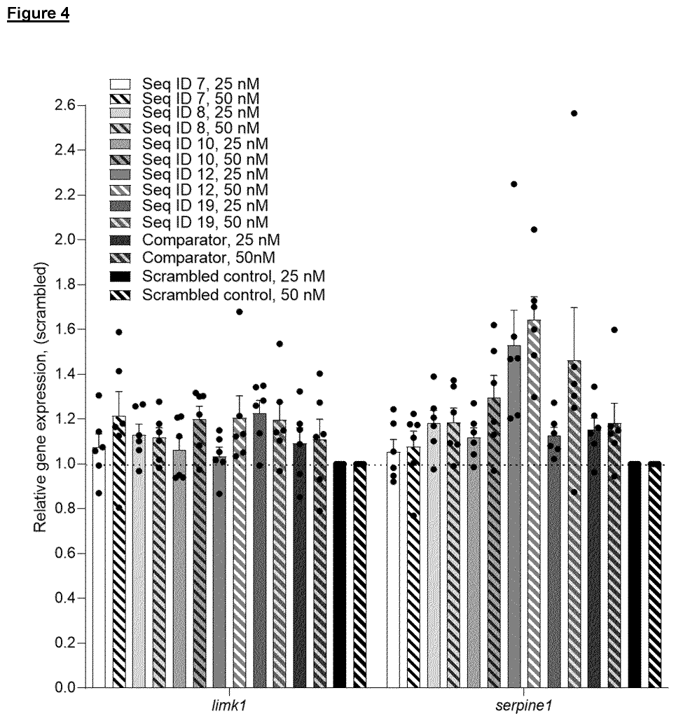

FIG. 4 depicts the levels of derepression of miR-134 target mRNA.

TABLE 2
qPCR primers and probes

| | Forward | Reverse | | |
|---|---|---|---|---|
| Gene | primer | primer | Probe | Cat.no: |
| Limk1 | GGTAGCAT | AGATGGAC | /56-FAM/TCCCAAAGC/ | Custom- |
| | CAGGTATT | ACGAAGCA | ZEN/AGAGAGAGAGA | made |
| | GACAGG | AGAG | GAGGT/3IABkFQ/ | |
| Serpine1 | CCTCTTGT | GATGGGCA | /56-FAM/TTCTTCCTT/ | Custom- |
| | ATCGTCCT | AGGTGTTC | ZEN/TGGCCTGGGTCAT | made |
| | CCATTG | ATTTC | CC/3IABkFQ/ | |
| Hprt1 | GGAGAACA | TGTGAAGT | /56-FAM/TGTTGACCC/ | Rn.PT. |
| | ATTCTGGG | TCCCCATA | ZEN/ACCAGCAGTTCAG | 39a. |
| | TTTGATC | AGGC | T/3IABkFQ/ | 22214832 |

### Example 6: miR-134 Target Protein Derepression in Cultured PC-12 Adh Cell Line

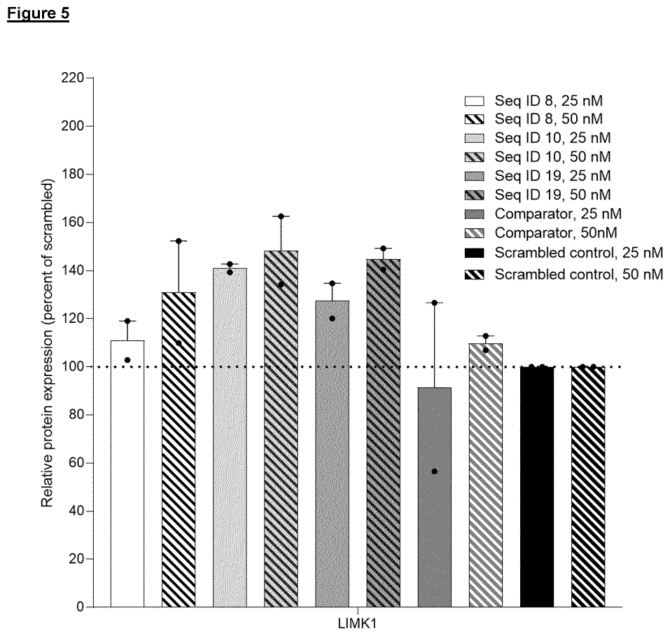

The transfection was done exactly as in example 5 except that the PC-12 Adh cells were seeded in 6-well CellBind plates (cat. no: CLS3335, Corning World, Corning, NY, USA) at 6.25×105 cells/well and 15 μL/well Lipofectamine 2000 (cat. no: 11668019, Thermo Fischer Scientific) was used. Forty-eight hours after transfection, cellular protein was extracted using RIPA buffer (cat. no: 89900, Thermo Fischer Scientific) supplemented with Complete™ Protease Inhibitor Cocktail (cat. no: 11697498001, Sigma-Aldrich) and protein concentration measured using Bio-Rad Protein Assay Kit II (cat. no: 5000002, Bio-Rad) as per manufacturer's instructions. For the electrophoresis, 24 μg protein was loaded on to a 12% gel (Criterion TGX stain-free, Bio-Rad) after which the proteins were blotted onto a PVDF membrane (Bio-Rad). The membrane was incubated with primary antibodies against LIMK1 (cat. no: 3842, Cell Signaling Technology, 1:500 in 5% BSA) and GAPDH (cat. no: 60004-1-Ig, Proteintech, 1:20,000 in EveryBlot Blocking Buffer (cat: 12010020, Bio-Rad)) overnight at 4° C. The following day, the secondary HRP-conjugated antibodies (donkey-a-rabbit, cat. no: 31458, 1:3,000 in 5% BSA and donkey-a-mouse, cat. no: SA1-100, 1:2,000 in EveryBlot Blocking Buffer; Thermo Fischer Scientific) were incubated for one hour at room temperature. Hereafter, the membrane was incubated with Clarity Western ECL substrate (cat. no: 1705061, Bio-Rad) for five minutes and then imaged on a ChemiDoc Mp Imager (Bio-Rad). The membranes were analysed in ImageStudio Lite (Li-Cor) and LIMK1 expression normalized to GAPDH and then expressed as percentage of the scrambled oligonucleotide control. As assay controls, a mouse brain protein sample and protein from cells not treated with miR-134 mimic were used. An example of an analysed membrane can be seen in FIG. 5.

### Example 7: Luciferase Assay of SEQ ID NO's 23-60 and 19 Antisense Oligonucleotides in Cultured PC-12 Adh Cells

This experiment was conducted as in example 2 with the following exceptions:

The antimiR oligonucleotides were transfected in concentrations of 0.5 and 5 nM in clear-bottom, white 96-well plates (cat. no 3610, Corning) treated with collagen (Sigma Aldrich cat. no. C8919). Background subtraction was not conducted and the Renilla intensity was normalized to Firefly and plotted against the ASO concentration.

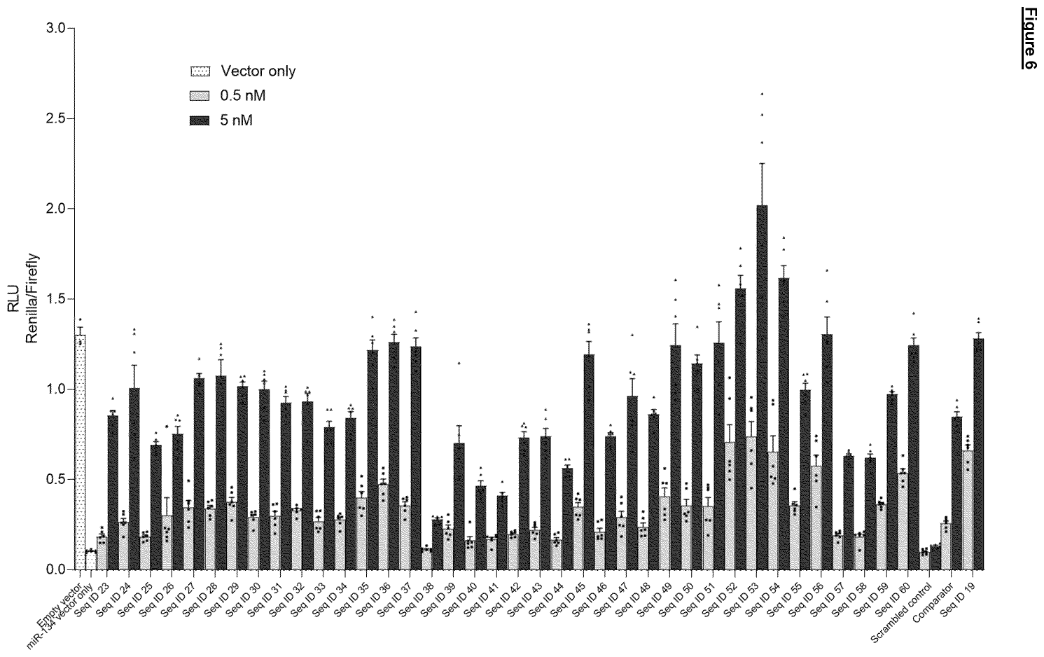

FIG. 6 shows the level of derepression of Renilla signal normalized to Firefly luminescence by the new antisense oligonucleotides compared with Seq ID 19 and comparator.

### Example 8: IC50 Determination in Cultured PC-12 Adh Cells

This experiment was conducted as in example 3 except it was conducted in clear-bottom, white 96-well plates (cat. no 3610, Corning) treated with collagen (Sigma Aldrich cat. no. C8919).

Background subtraction was not conducted and the Renilla intensity was normalized to Firefly and plotted against the ASO concentration.

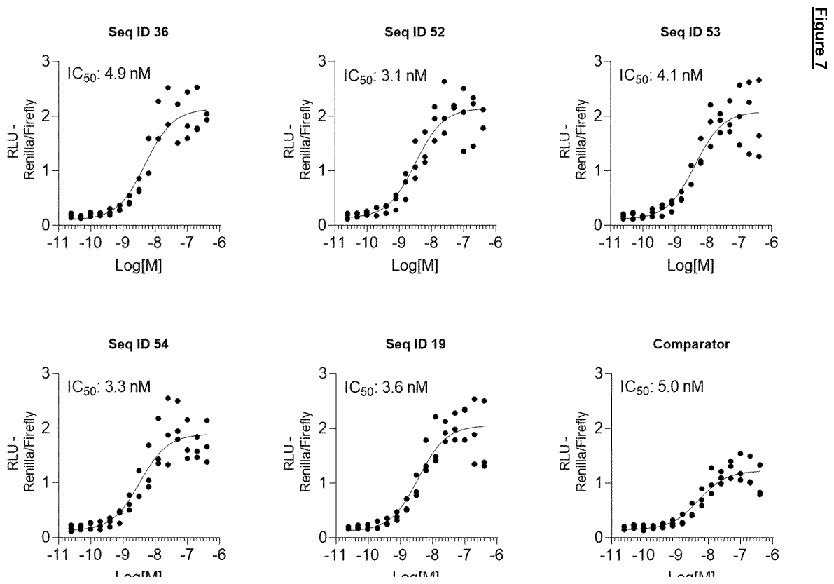

The dose-response curves and the IC50 values are depicted in FIG. 7.

### EMBODIMENTS

1) An antisense oligonucleotide complementary to miR-134 (SEQ ID NO: 1 or 2) comprising a sequence of 17-19 such as 18-19 nucleotides in length, wherein the antisense oligonucleotide is a mixmer having from seven to 14 such as from 7-12 such as from 10-13 such as from 10-11 affinity-enhancing nucleotide analogues and does not contain a stretch of more than three contiguous DNA nucleotides, and wherein said antisense oligonucleotide comprises one to 18 phosphorothioate internucleoside linkages.antisense oligonucleotide
2) The antisense oligonucleotides according to embodiment 1, wherein the antisense oligonucleotide is complementary to SEQ ID NO: 3.
3) The antisense oligonucleotide according to embodiment 1 or 2, which comprises the sequence SEQ ID NO: 4.
4) The antisense oligonucleotide according to any one of embodiments 1 to 3, wherein the antisense oligonucleotide is 18 or 19 nucleotides in length, comprises SEQ ID NO: 4, and is a LNA/DNA mixmer.
5) The antisense oligonucleotide according to any one of embodiments 1 to 4, wherein the antisense oligonucleotide is 17, 18 or 19 nucleotides in length, comprises SEQ ID NO: 4 and is a LNA/DNA mixmer having between 40 and 70% LNA, such as between 55 and 65% LNA, such as between 55 and 61% LNA, such as at least 50% LNA, such as at least 55% LNA.
6) The antisense oligonucleotide according to any one of embodiments 1 to 5, wherein the two terminal nucleotides in the 3′end are LNA or wherein the two terminal nucleotides in each end are LNA.
7) The antisense oligonucleotide according to any one of embodiments 1 to 6, wherein the LNA is Beta-D-Oxy LNA.
8) The antisense oligonucleotide according to any one of embodiments 1 to 7, wherein all the internucleoside bonds are phosphorothioate bonds.
9) The antisense oligonucleotide according to any one of embodiments 1 to 8, wherein the antisense oligonucleotide is anyone of SEQ ID NO's 5-21 or 23-60.
10) The antisense oligonucleotide according to any one of embodiments 1 to 9, wherein the antisense oligonucleotide is selected from any one of SEQ ID NO's 7, 8, 10, 12, 19, 24, 26-37, 45-56, 59 or 60, such as anyone of SEQ ID NO 19, 36, 52, 53, and 54, and wherein C is 5-methylcytosine, LNA is beta-D-oxy LNA and all internucleoside bonds are phosphorothioate bonds.
11) The antisense oligonucleotide according to anyone of embodiments 1 to 10, wherein the LNA/DNA mixmer further comprises one or more nucleosides that are anyone of tricyclo-DNA, 2′-Fluoro, 2′-O-methyl, 2′methoxyethyl (2′MOE), 2′ cyclic ethyl (CET), UNA, 2′fluoro and Conformationally Restricted Nucleoside (CRN).
12) The antisense oligonucleotide according to any one of embodiments 1 to 11, for use as a medicament.
13) A miR-134 inhibitory composition comprising the antisense oligonucleotides according to anyone of embodiments 1 to 12.
14) The antisense oligonucleotide for use as a medicament according to embodiment 12, or the composition according to embodiment 13, wherein the antisense oligonucleotide is anyone of SEQ ID NO's: 5-21 or 23-60.
15) The use or composition according to embodiment 12, 13 or 14, wherein the use is for the treatment, alleviation, amelioration, pre-emptive treatment or prophylaxis of a miR-134 related disease where modification of miR-134 activity is beneficial.
16) The use or composition according to embodiment 12, 13, 14 or 15, wherein the use is for the treatment, alleviation, amelioration, pre-emptive treatment or prophylaxis of a miR-134 related disease of the CNS or PNS.
17) The use according to embodiment 16, wherein the use is for treatment, alleviation, amelioration, pre-emptive treatment or prophylaxis of a neurological disorder.
18) The use according to embodiment 16 or 17, wherein the use is for treatment, alleviation, amelioration, pre-emptive treatment or prophylaxis of a neurodegenerative disorder.
19) The use according to embodiment 16, wherein the use is for treatment, alleviation, amelioration, pre-emptive treatment or prophylaxis of a neurodevelopmental disorder, a genetic disorder, and/or a genetic neurodevelopmental disorder.
20) The use according to anyone of embodiments 12 to 17, wherein the use is for treatment, alleviation, amelioration, pre-emptive treatment or prophylaxis of epilepsy.
21) The use according to embodiment 20, wherein the use is for treatment, alleviation, amelioration, pre-emptive treatment or prophylaxis of drug resistant epilepsy.
22) The use according to embodiment 20, wherein the use is for treatment, alleviation, amelioration, pre-emptive treatment or prophylaxis of seizures in epilepsy.
23) The use according to embodiment 22, wherein the use is for treatment, alleviation, amelioration, pre-emptive treatment or prophylaxis of spontaneous seizures in epilepsy.
24) The use according to embodiment 22 or 23, wherein the use is for treatment, alleviation, amelioration, pre-emptive treatment or prophylaxis of therapy resistant seizures.
25) The use according to embodiment 20 to 23, wherein said epilepsy is a focal epilepsy, preferably wherein said focal epilepsy is focused in the frontal lobe, the parietal lobe, the occipital lobe or the temporal lobe.
26) The use according to embodiment 20 to 23, wherein said epilepsy is a generalised epilepsy, preferably wherein said generalised epilepsy is selected among absences, myoclonic seizures, tonic-clonic seizures, tonic seizures, atonic seizures, clonic seizures and spasms.
27) The use according to embodiment 20 to 26, wherein said epilepsy is status epilepticus.
28) The use according to embodiment 20 to 27, wherein said epilepsy is selected among autosomal dominant nocturnal frontal lobe epilepsy, continuous spike-and-waves during slow sleep, Dravet syndrome, epilepsy developed after apoplexy, epileptic encephalopathy, Gelastic epilepsy, absences, benign neonatal seizures, Jeavons syndrome, Juvenile myoclonic epilepsy, Landau-Kleffner Syndrom, Lennox-Gastaut syndrome, Mesial temporal lobe epilepsy, myoclonic astatic epilepsy, Ohtahara Syndrom, Panayiotopoulos syndrome, PCDH19 syndrom, benign childhood epilepsy with centrotemporal spikes, Sturge-Weber syndrome, symptomatic focal epilepsy, transient epileptic amnesia and West syndrome.
29) The use according to embodiment 20 to 28, wherein said epilepsy is present together with a comorbidity selected among a psychiatric disorder, a cognitive disorder, a sleep disorder, a cardiovascular disorder, a respiratory disorder, an inflammatory disorder, anxiety, pain, cognitive impairment, depression, dementia, headache, migraine, heart disease, ulcers, peptic ulcers, arthritis and osteoporosis.
30) The use according to embodiments 16 to 29, wherein the use is for prevention or prophylaxis, amelioration, or alleviation or treatment of neuronal damage.
31) The use according to embodiment 30, wherein the use is for treatment, alleviation, amelioration, pre-emptive treatment or prophylaxis of hippocampal damage.
32) The use according to embodiment 16, wherein the use is for treatment, alleviation, amelioration, pre-emptive treatment or prophylaxis of oxidative stress, inflammation and/or apoptosis.
33) The use according to embodiment 16, wherein the use is for treatment, alleviation, amelioration, pre-emptive treatment or prophylaxis of intracerebral hemorrhage-induced brain injury, ischemic stroke, haemorrhagic stroke or stroke
34) The use according to embodiment 16 to 19, wherein the use is for treatment, alleviation, amelioration, pre-emptive treatment or prophylaxis of an autoimmune disease, a memory disorder, hippocampal sclerosis, Parkinsons Disease, a demyelinating disease, multiple sclerosis, spinal cord injury, acute spinal cord injury, amyotrophic lateral sclerosis, progressive bulbar palsy, progressive muscular atrophy, primary lateral sclerosis, ataxia, bell's palsy, a hereditary neurological disease, Charcot-Marie-Tooth, a headache, Horton's headache, migraine, pick's disease, progressive supranuclear palsy, multi-system degeneration, motor neuron diseases, Huntington's disease, prion disease, Creutzfeldt-Jakob disease, corticobasal degeneration, aphasia, primary progressive aphasia, a movement disorder or symptoms or effects thereof.
35) The use according to embodiment 16 to 19, wherein the use is for treatment, alleviation, amelioration, pre-emptive treatment or prophylaxis of dementia.
36) The use according to embodiment 35, wherein said dementia is selected among Alzheimer disease, vascular dementia, frontotemporal dementia and Lewy bodies dementia.
37) The use according to embodiment 12 or any one of 14 to 19, wherein the use is for treatment, alleviation, amelioration, pre-emptive treatment or prophylaxis of pain.
38) The use according to any of embodiments 12 to 16, wherein the use is for treatment, alleviation, amelioration, pre-emptive treatment or prophylaxis of a psychiatric disease wherein modulation of miR-134 is beneficial.
39) The use according to embodiment 38, wherein the use is for treatment, alleviation, amelioration, pre-emptive treatment or prophylaxis of autism, or a mood disorder, depressive disorder, schizophrenia, bipolar disorder, attention deficit hyperactivity disorder, anxiety or Tourette.
40) The use according to embodiment 39, wherein the use is for treatment, alleviation, amelioration, pre-emptive treatment or prophylaxis of major depressive disorder.
41) The use according to embodiment any one of 12 to 17, wherein the use is for treatment, alleviation, amelioration, pre-emptive treatment or prophylaxis of a memory disorder, wherein modulation of miR-134 expression or activity is beneficial.
42) The use according to embodiment any one of 12 to 15, wherein the use is for treatment, alleviation, amelioration, pre-emptive treatment or prophylaxis of a cancer.
43) The use according to embodiment 42, wherein said cancer is a cancer in the nerve system, preferably glioma.
44) The use according to embodiment 42, wherein the use is for treatment, alleviation, amelioration, pre-emptive treatment or prophylaxis of cancer selected from the group of lung tumors, non-small cell lung cancer, glioma, head and neck squamous cell carcinoma, pancreatic cancer, colon cancer, prostate cancer, melanoma, uveal melanoma, oral squamous cell carcinoma or squamous cell carcinoma of the tongue.
45) The use according to any one of embodiments 12 to 17, wherein the antisense oligonucleotide is for use in treating anyone of Prader-Willis Syndrome, Anglemans Syndrome, a cardiovascular disorder, atherosclerosis, and pulmonary disease.
46) The use according to embodiment 12 or any one of 14 to 19, wherein the use is for treatment, alleviation, amelioration, pre-emptive treatment or prophylaxis of an infection.
47) The use according to embodiment 46, wherein said infection is selected among, sepsis, meningitis and encephalitis.
48) The use according to any one of embodiment 12 or 14 to 19, wherein said use is for treatment, alleviation, amelioration, pre-emptive treatment or prophylaxis of a genetic disorder, preferably neurofibromatosis.
49) The use according to anyone of embodiments 12 to 48, wherein the antisense oligonucleotide according to anyone of embodiments 1 to 11 of the invention is for use in combination with one or more other therapies.
50) The use according to embodiment 48, wherein said therapy is an antisense oligonucleotide complementary to miR-27b.
51) The use according to embodiment 49, wherein said therapy is an antisense oligonuleotide complementary to adenosine kinase mRNA or another adenosine kinase inhibitor.
52) The use according to embodiment 49, wherein said therapy induces the Nrf-2/ARE pathway in a mammal, such as in a human.
53) The use according to embodiment 49, wherein said therapy is one or more of an antisense oligonucleotide complementary to miR-27b, an antisense oligonucleotide complementary to adenosine kinase mRNA and a therapy inducing the Nrf-2/ARE pathway.
54) The use according to any one of embodiment 12 to 48, wherein the antisense oligonucleotides according to anyone of embodiment 1 to 12 is the sole active pharmaceutical ingredient.
55) A pharmaceutical composition comprising an effective dosage of the antisense oligonucleotide according to anyone of embodiments 1 to 12 and a pharmaceutically acceptable carrier.
56) A pharmaceutical composition comprising an effective dosage of the antisense oligonucleotide according to anyone of embodiments 1 to 12, wherein said antisense oligonucleotide is the sole active pharmaceutical ingredient.
57) The pharmaceutical composition according to embodiment 49, wherein the composition is for use according to any one of embodiments 12 to 49.
58) The pharmaceutical composition according to embodiments 55 or 57, wherein the composition is for administration by subcutaneous administration, intravenous administration, parenteral administration, nasal administration, pulmonary administration, rectal administration, vaginal administration, intrauterine administration, Intraurethral administration, administration to the eye, administration to the ear, cutaneous administration, intradermal administration, intramuscular administration, intraperitoneal administration, epidural administration, intraventricular administration, intracerebral, intrathecal administration or oral administration or for administration directly into the brain or cerebrospinal fluid, or wherein said composition is administered as an implant.
59) The pharmaceutical composition according to embodiment 55 or 56, wherein the composition is for intratecal administration.
60) The pharmaceutical composition according to embodiment 55 or 56, wherein the composition is for intracerebroventricular administration.
61) The pharmaceutical composition according to embodiment any one of 55 to 58, wherein said composition is administered in a pump, preferably wherein said pump is a mini pump, more preferably wherein said mini pump is a mini-osmotic pump.
62) The pharmaceutical composition according to embodiment any one of 55 to 61, wherein said composition is for intraventricular administration facilitated by an intraventricular catheter, preferably wherein said catheter is attached to a reservoir, preferably wherein said reservoir is an Ommaya reservoir.
63) The pharmaceutical composition according to embodiment any one of 55 to 62, wherein said composition is administrated with an interval of 1 day, 2 days, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or preferably 120 days.
64) The pharmaceutical composition according to embodiment any one of 55 to 63, wherein said composition is administrated with an interval of between 1-200 days, 10-190 days, 20-180 days, 30-170 days, 40-160 days, 50-150 days, 60-140 days, 70-130 days, 80-120 days, 90-110 days or preferably about 100 days.
65) The antimiR-134 oligonucleotide according to any one of embodiments 1 to 12 or the composition according to embodiment 13 for use in a method of treating the diseases according to any one of embodiments 12 to 49.
66) A method of treatment of the diseases according to any one of embodiments 12 to 49 by use of the antisense oligonucleotides according to any one of embodiments 1 to 12 or the composition according to embodiment 13.
67) The use according to any one of embodiments 12 to 49, or method according to any one of embodiments 48 to 65, wherein the treatment is anyone of preventive, curative or disease modifying.
68) A method of diagnosing a disease according to any one of embodiments 12 to 49 by use of the antisense oligonucleotide according to any one of embodiments 1 to 12 or the composition according to embodiment 13.

### REFERENCES

1) Jimenez-Mateos et al., Nature Medicine (2012) volume 18, pages 1087-1094.

1. An antisense oligonucleotide comprising a sequence of 17-19 nucleotides in length complementary to miR-134, wherein the antisense oligonucleotide is a mixmer having from seven to 14, affinity-enhancing nucleotide analogues and does not contain a stretch of more than three contiguous DNA nucleotides, and wherein said antisense oligonucleotide comprises one to 18 phosphorothioate internucleoside linkages.

2-15. (canceled)

16. The antisense oligonucleotides according to claim 1, wherein the antisense oligonucleotide is complementary to SEQ ID NO: 3.

17. The antisense oligonucleotide according to claim 1, which comprises the sequence set forth by SEQ ID NO: 4.

18. The antisense oligonucleotide according to claim 1, wherein the antisense oligonucleotide is 18 or 19 nucleotides in length, comprises the sequence set forth by SEQ ID NO: 4 and, wherein the antisense oligonucleotide is a LNA/DNA mixmer.

19. The antisense oligonucleotide according to claim 1, wherein the antisense oligonucleotide is 18 or 19 nucleotides in length, comprises the sequence set forth by SEQ ID NO: 4 and, wherein between 40 and 70% of the nucleosides of said mixmer is LNA.

20. The antisense oligonucleotide according to claim 1, wherein the two terminal nucleotides in the 3′end are LNA, or wherein the two terminal nucleotides in each end are LNA.

21. The antisense oligonucleotide according to claim 1, wherein the LNA is Beta-D-Oxy LNA, and LNA cytosines are 5-methylcytosine.

22. The antisense oligonucleotide according to claim 1, wherein all the internucleoside bonds are phosphorothioate bonds.

23. The antisense oligonucleotide according to claim 1, wherein the antisense oligonucleotide is anyone of the sequences set forth by SEQ ID NO's 19, 5-18, 20, 21 or 23-60.

24. The antisense oligonucleotide according to claim 23, wherein the antisense oligonucleotide is anyone of SEQ ID NO's:

| (SEQ ID NO 19) | |
|---|---|
| | 5′ CCTctGgTcAAccAGtcAC 3′ |
| (SEQ ID NO 36) | |
| | 5′ CctctGgTcAAccAgTcAC 3′ |
| (SEQ ID NO 52) | |
| | 5′ CCTctGgTcAAccAgTcAC 3′ |
| (SEQ ID NO 53) | |
| | 5′ CCTctGgTcAAccaGTcAC 3′ |
| | or |
| (SEQ ID NO 54) | |
| | 5′ CTCtgGTcaAccAgTcAC 3′ |

and, wherein capital letters are LNA, small letters are DNA, capital C is 5-methylcytosine, the LNA is beta-D-oxy LNA and all internucleoside bonds are phosphorothioate bonds.

25. The antisense oligonucleotide according to claim 1, wherein the mixmer is a LNA/DNA mixmer further comprising one or more nucleosides that are anyone of tricyclo-DNA, 2′-Fluoro, 2′-O-methyl, 2′methoxyethyl (2′MOE), 2′ cyclic ethyl (CET), UNA, 2′fluoro or Conformationally Restricted Nucleoside (CRN).

26. A pharmaceutical composition comprising an effective dosage of the antisense oligonucleotides of claim 1.

27. A method for the treatment, alleviation, amelioration, pre-emptive treatment or prophylaxis treatment of a miR-134 related disease of the CNS or PNS, said method comprising administrating to a subject in the need thereof an antisense oligonucleotide comprising a sequence of 17-19 nucleotides in length complementary to miR-134, wherein the antisense oligonucleotide is a mixmer having from seven to 14, affinity-enhancing nucleotide analogues and does not contain a stretch of more than three contiguous DNA nucleotides and, wherein said antisense oligonucleotide comprises one to 18 phosphorothioate internucleoside linkages.

28. The method according to claim 27, wherein the disease of the CNS or PNS is a neurological disorder.

29. The method according to claim 27, wherein the disease of the CNS or PNS is epilepsy.
